(19) United States
(12) Patent Application Publication
(10) Pub. No.: US 2018/0361046 A1
(43) Pub. Date: Dec. 20, 2018

(54) Hemodialysis Supply Box and Drain Cabinet for Continuous Renal Replacement Therapy

(71) Applicant: HKS, Inc., Dallas, TX (US)

(72) Inventors: Camilla Moretti, Northville, MI (US); Alexandra Bemetich, Northville, MI (US); Kelly Miller, Atlanta, GA (US)

(21) Appl. No.: 16/011,201

(22) Filed: Jun. 18, 2018

Related U.S. Application Data
(60) Provisional application No. 62/521,842, filed on Jun. 19, 2017.
(60) Provisional application No. 62/527,105, filed on Jun. 30, 2017.

(51) Int. Cl. A61M 1/16; A61M 1/34

(52) U.S. Cl. CPC A61M 1/1621; A61M 1/1601; A61M 1/1694; A61M 2209/082; A61M 1/1668; A61M 1/341

(57) ABSTRACT

A wall-mounted hemodialysis supply box and drain cabinet for use in continuous renal replacement therapy is provided that comprises a corrosion-resistant enclosure defining an interior space having a forwardly facing opening with a hinged door, at least one water-supply valve, at least one variable height bag holder, a bottom wall with a drain, and a plurality of inclined inside walls configured to channel waste water and treatment fluids into the drain. A sprayer hose attachment for use in washing down and cleaning the interior space is also disclosed.

### CROSS-REFERENCE TO RELATED APPLICATIONS

This application claims the benefit of U.S. Provisional Application No. 62/521,842, filed Jun. 19, 2017 and U.S. Provisional Application No. 62/527,105, filed Jun. 30, 2017.

### TECHNICAL FIELD

This invention relates to a hemodialysis supply box and drain cabinet that is useful to healthcare workers performing hemodialysis or continuous renal replacement therapy (CRRT) on a patient in a hospital, intensive care unit or other medical care facility or dialysis center within which such procedures are performed. In various embodiments of the invention, the subject supply box and cabinet is fabricated from stainless steel or another similarly effective material and is mountable in or on a wall of a patient care area within a medical treatment facility. The invention preferably comprises at least one water inlet valve, at least one fluid drain through a bottom portion of the cabinet, at least one wash-down hose for use in flushing the interior of the cabinet, a fluid shield extending above and in front of the fluid drain, at least one adjustable bag holder disposed inside the cabinet, and a forwardly facing door through which the interior of the subject supply box and draining cabinet can be accessed by healthcare personnel before, during or after treatment.

### BACKGROUND

Many patients with medical conditions involving non-performing or under-performing kidneys require intermittent hemodialysis (IHD) or CRRT for survival. Continuous dialysis therapies are discussed and explained, for example, in “Continuous Dialysis Therapies: Core Curriculum 2016,” American Journal of Kidney Disease 2016; 68(4):645-657.

In the past, CRRT poly bags were often hung above the patient's toilet with flexible plastic drain tubing draped into the toilet. The toilet remained unusable for the duration of the drainage, and if the tubing inadvertently became disconnected from the bag, resultant splashing, spillage, contamination of healthcare workers, and even accidental flushing of tubing sometimes occurred. Manufacturers have offered small stainless steel supply and waste dialysis boxes comprising one or two inlet valves, a drain and a hinged door with a closure. Such boxes are not, however, known to be sufficiently large to accommodate and adjustably support poly bags of the type used to administer, mix, infuse or recover waste fluids during hemodialysis or CRRT and do not have an adjustable bag holder or a metal shield extending upwardly around the drain to reduce the likelihood of spillage or the possible splashing of treatment or waste fluids either during such procedures or later, during cleanup.

### SUMMARY OF THE INVENTION

The invention provides a healthcare worker in a medical treatment facility with a hemodialysis water connection and discharge capability, and with a safe, clean and efficient apparatus for use in receiving, adjustably supporting, draining and removing bags containing treatment liquids or waste fluids during CRRT. The subject hemodialysis supply box and CRRT drain cabinet is desirably configured to receive and support one or more bags of liquids used in such therapies that may contain chemicals potentially harmful to healthcare workers or persons other than the patient for whom they are intended. At least one inlet valve is provided that is desirably connected to a clean water source. Optionally, either that or another water valve can also be configured for attachment to a flexible hose fitted with a wand or sprayer controllable by a user for use during cleanup.

A hemodialysis supply box and drain cabinet for CRRT is disclosed that is desirably recessed into and flush-mounted in a wall of a medical treatment facility where such procedures are performed. The body of the device is preferably made of stainless steel or another similarly effective, corrosion-resistant metal alloy or durable polymeric material and is desirably configured to form an enclosure with at least one hemodialysis water connection, a height-adjustable bag hanger, and a bottom drain disposed in fluid communication with a waste disposal line or other suitable waste collection receptacle. Alternatively, for locations or situations where the subject apparatus is needed less often, the subject supply box and cabinet can be configured for mounting on a carriage or dolly supported by wheels to facilitate movement among different rooms or stations within a medical treatment facility provided that connections to water supply and sewer or other waste disposal flow lines are available.

In one embodiment, the invention is a prefabricated metal alloy box-shaped cabinet that is mountable in or on a wall in a medical treatment facility. The supply box and cabinet of the invention is preferably made primarily of stainless steel or another material that is resistant to corrosion or degradation when contacted by chemicals and compositions of the type used in or produced by such procedures. At least one, and preferably two, valves connected to water supply lines are desirably fitted inside the cabinet and a fluid drain is provided at the bottom that is connected to a sewer line or other suitable waste receptacle. Alternatively, at least one metering valve can be provided in place of one of the quarter-turn valves for use in supplying water at a more precisely controlled flow rate and volume for use during a procedure.

The top, bottom, side and back walls of the subject supply box and cabinet preferably define an interior space that is sufficiently large to accommodate and hang one or more bags of treatment fluids and also accommodate and supply water to one or more flexible hoses and an optional sprayer device if desired. In one embodiment, the supply box or cabinet comprises a forwardly facing shield configured as a front wall section that cooperates with the back, side and bottom walls to form a fluid-tight basin around and over a drain disposed in the bottom wall. The front wall section desirably helps shield healthcare workers, the patient and others from any treatment fluid or waste fluid that might otherwise spill out of the cabinet or splash onto or contaminate the patient or a healthcare worker. A forwardly facing opening is desirably provided in the front-facing wall of the device through which supplies such as poly bags containing treatment fluids can be received and supported inside the interior space of the supply box and drain cabinet.

A hinged door is desirably provided that is configured to selectively close the front opening of the supply box or drain cabinet whenever access to the interior space is not required. The door can be hinged along a side or bottom edge, for example, with one side of the hinge also being attached to one of the wall panels of the device. A magnetic latch or a mechanical latch or lock are desirably provided and cooperatively configured and aligned for selectively holding the door open or closed as desired. Additional portals, recesses, cutouts, grommets or other fittings can also be provided if needed for use in passing fluid flowlines (made of medically acceptable polymeric tubing) into or out of the supply box and drain cabinet if desirable when the door is closed.

A height-adjustable bag hanger is preferably attached to a top, back or side wall. In one embodiment of the invention, the height-adjustable bag hanger comprises a plate mounted in spaced-apart relation to the back wall of the device, an array of vertically and horizontally spaced-apart orifices in the plate, and a plurality of selectively repositionable hangers or hooks that are selectively and releasably engageable with the orifice plate and are also releasably engageable with one or more poly bags of treatment or waste fluids inside the device as needed.

The lower portion of the interior space defined by the walls and door of the supply box and cabinet preferably further comprises inclined wall sections that are cooperatively configured to channel any free liquid inside the cabinet toward and into the drain. The drain desirably functions in cooperation with such structural elements to direct any waste or fugitive liquids out of the supply box or cabinet and into a sewer line or other flow line that is part of a liquid waste recovery system. The drain is desirably or necessarily supplemented by a trap, trap primer or other mandated device intended to prevent backflow of sewer gas or liquids upwardly through the drain and into the supply box or cabinet.

### BRIEF DESCRIPTION OF THE DRAWINGS

The apparatus of the invention is further described and explained in relation to the following drawings wherein:

FIG. 1 is a simplified front elevation view of one embodiment of an uninstalled supply box and drain cabinet of the invention with a door shown in an open position;

FIG. 2 is a simplified, cross-sectional side elevation view of an embodiment of the invention as in FIG. 1 that is recessed into a wall;

FIG. 3 is a simplified front elevation view of a different embodiment of the invention wherein the door is hinged at the bottom of the front opening, wherein the valves are not shown to simplify the drawing, and wherein a poly bag containing treatment fluid is hanging from a height-adjustable hook disposed inside the interior space of the invention; and

FIG. 4 is a front elevation view of another embodiment of the invention in which the door and its hinge are not shown and wherein a wash-down hose with a hand-held sprayer device is installed on one water inlet valve of the invention.

The subject drawings are not to scale and some elements of the invention are omitted in some views to simplify the drawings for purposes of clarity and to facilitate reproduction of the drawings.

### DESCRIPTION OF PREFERRED EMBODIMENTS

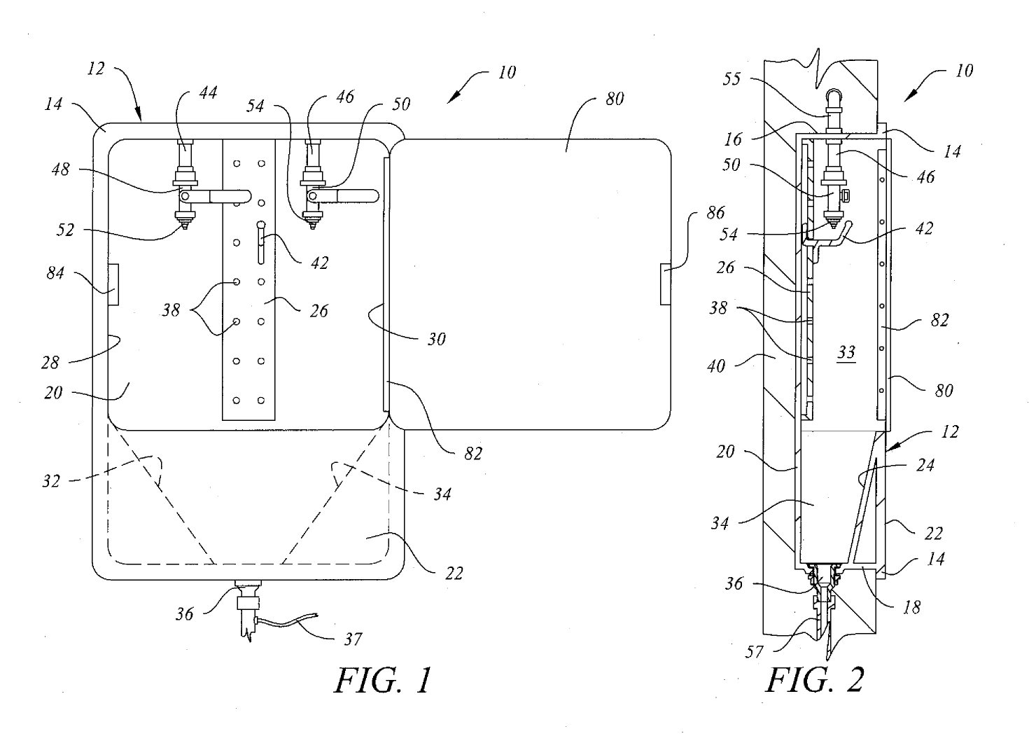

Referring to an embodiment of the invention as depicted in FIGS. 1-2, hemodialysis supply box and CRRT drain cabinet 10 (hereafter “cabinet 10”) comprises body 12 having a partially open front surrounded by flange 14. As shown in FIG. 2, the back side of flange 14 abuts against a forwardly facing surface of wall 40 whenever cabinet 10 is installed inside a recess in wall 40. As depicted in this embodiment, body 12 is a substantially rectangular box that further comprises top wall 16, bottom wall 18, back wall 20, side walls 28, 30, and front wall 22 that shields drain 36 beneath door 80. Inclined interior walls 24, 32, 34 satisfactorily extend as high as the top of front wall 22 and are intended to channel water, treatment fluids, waste fluids or other liquids flowing downwardly through interior space 33 (FIG. 2) toward drain 36 disposed in bottom wall 18. If needed, trap primer 37 or another similarly effective device can also be provided beneath drain 36 to prevent waste gas or fluids from flowing back into cabinet 12. Front wall 22 cooperates with inclined interior wall 24 to shield users by blocking liquid that might otherwise spill or splash out of cabinet 10 prior to reaching drain 36 whenever door 80 is open.

Body 12 and door 80 cooperate to define interior space 33 (FIG. 2) inside cabinet 10. Door 80 is attached to side wall 30 by continuous hinge 82 (FIG. 2) and closure 84, 86 (FIG. 1) is provided for use in latching door 80 in a closed position when desired. It will be appreciated by those of ordinary skill in the art upon reading this disclosure that other similarly effective hinges and latching devices can also be used within the scope of the invention to assist in maintaining door 80 in either an open or closed position. Optionally, door 80 can be opaque, transparent or translucent and made of metal, glass or a durable polymeric material suitable for use in a medical environment. Similarly, door 80 can be made of an opaque material with a transparent viewing window comprising either a major or minor portion of the available surface area of the door.

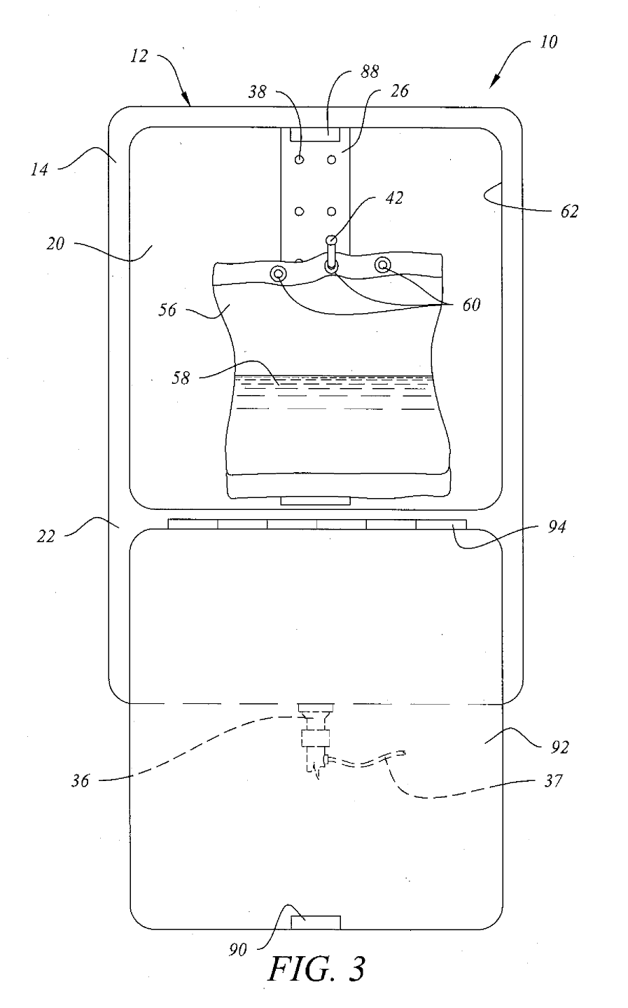

Still referring to FIGS. 1-2, the height-adjustable bag holder of the invention is depicted in this embodiment as comprising an elongated metal plate 26 having rearwardly facing flanges along the top and bottom edges that are used to mount plate 26 in spaced-apart relation to back wall 28 of body 12. An array of vertically and horizontally spaced-apart orifices 38 desirably extends through plate 26 to facilitate insertion of one or more hangers 42 that, for example, can be configured substantially as shown in FIG. 2. Hangers 42 as shown in this embodiment are configured similarly to hooks used to support objects on conventional pegboard in non-medical environments, although other differently configured repositionable hangers that are capable of supporting one or more poly bags containing CRRT treatment liquids or fluids can likewise be used within the scope of the invention if made of suitable materials and with possible associated modifications to metal plate 26 as needed to achieve comparable functionality for use in this device and application. In this embodiment, however, metal plate 26 and hangers 42 are desirably made of stainless steel or another similarly durable and corrosion-resistant material. Depending upon the size of the bag(s) being used and upon the total weight to be supported by the adjustable bag holder, a plurality of hangers 42 can be used to support each bag. Referring to FIG. 3, poly bag 56 containing liquid 58 is supported inside cabinet 10 by inserting the forwardly facing and upturned tip of hanger 42 through one of grommets 60 disposed along the upper edge of bag 56.

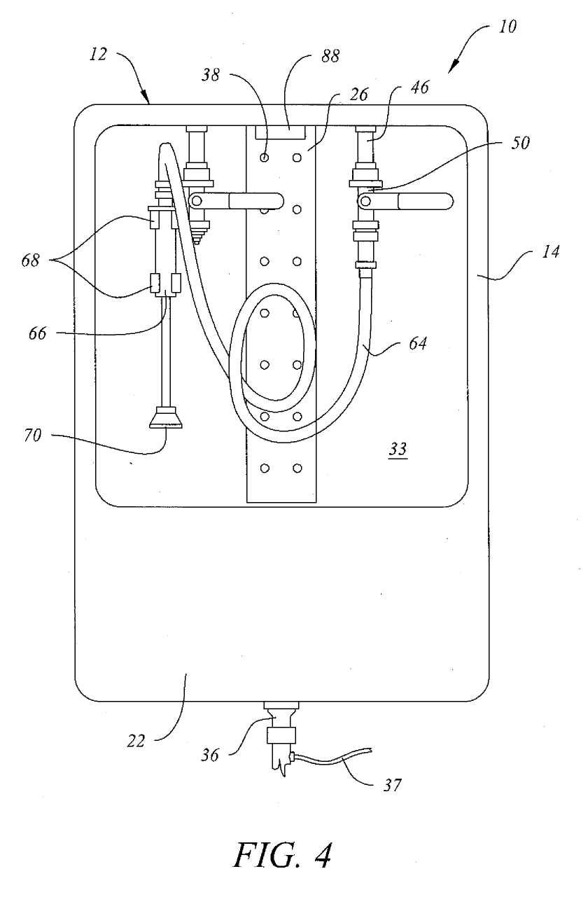

Still referring to FIGS. 1-2, water inlet lines 44, 46 and quarter-turn valves 48, 50 installed inside top wall 16 of body 12 can be connected to in-wall water supply lines such as line 55 shown in FIG. 2 for the purpose of providing water to cabinet 10 for various possible uses in relation to hemodialysis and CRRT procedures and in associated wash-down or clean-up of cabinet 10 or for other ancillary medical purposes. As shown in FIGS. 1-2, the fluid outlets of valves 48, 50 are each provided with male fittings configured as hose barbs for insertion into a cooperatively configured end of a segment of flexible hose or tubing. Referring to FIG. 4, a threaded coupling is provided for use in attaching flexible hose 64 to valve 50. Although quarter-turn valves are shown because they can be quickly opened and closed, it will be appreciated that other similarly effective water valves can also be used with cabinet 10 of the invention. Also in FIG. 4, support brackets 68 are attached to back wall 20 of cabinet 10 to support a wand or sprayer 66 with nozzle 70 that can be used to wash down interior space 33 of cabinet 10 following draining and removal of hanger 42 and a bag or bags such as poly bag 56 shown in FIG. 3 at the completion of a procedure or other use of cabinet 10 (such as, for example, cleaning out a bedpan used by a patient undergoing hemodialysis or CRRT. It should be noted that valves 44, 46 as shown in FIGS. 1-2, 4 are not shown in FIG. 3 in order to more clearly illustrate hanging of a bag such as bag 56 on the height-adjustable bag holder of the invention. Similarly, door 92 and hinge 94 as shown in FIG. 3 are not shown in FIG. 4 in order to better illustrate attachment of flexible hose 64 to valve 50.

Other alterations and modifications of the invention will likewise become apparent to those of ordinary skill in the art upon reading this specification in view of the accompanying drawings, and it is intended that the scope of the invention disclosed herein be limited only by the broadest interpretation of the appended claims to which the inventors and/or Applicant are legally entitled.

What is claimed is:

1. A hemodialysis supply box and CRRT drain cabinet, comprising:
a wall-mounted enclosure comprising corrosion-resistant top, bottom, front, back and side walls;
the front wall further comprising a forwardly facing opening with a hinged door and a liquid shield disposed below the opening;
at least one water inlet valve;
a drain disposed in the bottom wall;
a plurality of inclined interior wall sections disposed above and around the drain; and
at least one height-adjustable bag holder.

2. The hemodialysis supply box and CRRT drain cabinet of claim 1 wherein the top, bottom, front, back and side walls and the inclined interior wall sections are made of a medically acceptable material.

3. The hemodialysis supply box and CRRT drain cabinet of claim 2 wherein the medically acceptable material is a metal alloy.

4. The hemodialysis supply box and CRRT drain cabinet of claim 3 for use in continuous renal replacement therapy, wherein the medically acceptable material is stainless steel.

5. The hemodialysis supply box and CRRT drain cabinet of claim 1 comprising two water inlet valves.

6. The hemodialysis supply box and CRRT drain cabinet of claim 1 wherein the height-adjustable bag holder comprises a metal plate spaced apart from the back wall, the metal plate further comprising an array of spaced-apart orifices.

7. The hemodialysis supply box and CRRT drain cabinet of claim 1 wherein the height adjustable bag holder comprises at least one bag hanger that is releasably engageable with a metal plate spaced apart from the back wall.

8. The hemodialysis supply box and CRRT drain cabinet of claim 1 further comprising a flexible hose.

9. The hemodialysis supply box and CRRT drain cabinet of claim 8 further comprising a sprayer disposed in fluid communication with the flexible hose.

10. The hemodialysis supply box and CRRT drain cabinet of claim 1 further comprising a trap primer disposed beneath the drain.

11. The hemodialysis supply box and CRRT drain cabinet of claim 1 comprising a door hinge disposed beneath the forwardly facing opening.

12. The hemodialysis supply box and CRRT drain cabinet of claim 1 comprising a door hinge disposed at one side of the forwardly facing opening.
